(19) United States
(12) Patent Application Publication
(10) Pub. No.: US 2008/0000870 A1
(43) Pub. Date: Jan. 3, 2008

(54) STOPPER FOR FLASKS OF STERILE PRODUCTS AND USE OF SAID STOPPER IN STERILE MEASURED FILLING

(75) Inventors: Diego Lopez Alvarez, La Garriga (Barcelona) (ES); Sergi Roura Adell, Arenys de Munt (Barcelona) (ES); Jose Ramon Sanchez Sabate, Parets del Valles (Barcelona) (ES)

Correspondence Address: DARBY & DARBY P.C., P.O. BOX 770, Church Street Station, New York, NY, 10008-0770, US

(73) Assignee: GRIFOLS, S.A., Barcelona (ES)

(21) Appl. No.: 11/759,578

(22) Filed: Jun. 7, 2007

(30) Foreign Application Priority Data
Jun. 19, 2006 (ES) 200601691

(51) Int. Cl. B65D 39/00; B65D 51/00; A61B 19/00

(52) U.S. Cl. 215/355; 215/247; 604/415

(57) ABSTRACT

A stopper for flasks for pharmaceutical products, characterized in that it comprises, in combination: a plug with resilient means for adjustment to the inner face of the neck and of the mouth of the container and an upper stoppering wall; means for defining a position for pre-stoppering of the flask, before the stoppered position; a protector joined to the plug which comprises a lateral wall of synthetic material, said lateral wall having a projection for engagement with an annular protuberance located on the mouth of the container; the aforesaid protector having an extension which, when the stopper is in its pre-stoppered position, creates a labyrinth closure with respect to the particles which might be present in the surroundings in which the container is being handled.

### CROSS-REFERENCE TO RELATED APPLICATIONS

This application claims priority to Spanish patent application Ser. No. 200601691 filed Jun. 19, 2006, the contents of which are hereby incorporated by reference in their entirety.

### DESCRIPTION

The present invention relates to a stopper for flasks, especially for flasks of sterile products, and to the use of same in a method of sterile measured filling of flasks.

More particularly, the stopper of the present invention is especially suitable as a stopper for vials the sterile contents of which are sealed and are to be administered by injection.

However, specific embodiments of the present invention are not necessarily confined to the applications cited.

The documents ES2016490 and ES2232269 disclose a method of sterile measured filling of flasks, preferably of vials, which comprises the steps of placing a stopper on a flask in a pre-stoppered position, placing a protector thereon, sterilising the flask in an autoclave with the stopper and the protector in a pre-closed position, cooling the flask-stopper-protector assembly, removal of the protector and the stopper, measured filling of the contents of the container into the latter, and closing the flask with the stopper. During the process, the protector and the stopper exert a labyrinth closure action which permits the sterilisation of the interior of the container in the autoclave, but prevents the entry of particles which might be present in the non-sterile air.

After the measured filling, the stopper is inserted fully into the mouth of the container. Since the stopper includes a metal capsule, the final sealing of the container is obtained by deformation of said capsule (encapsulation or beading of the capsule). In accordance with the research carried out by the Applicant, the skirt of the protector and the design of the stopper improve the sterility of the whole owing to the labyrinth closure effect which is established between the skirts or walls of the protector and the wall of the flask or vial.

It is an aim of the present invention to disclose means which permit the simplification, reduction of costs and increase in safety in the aforesaid method.

For this purpose, the present invention discloses a stopper for a flask, especially a flask for pharmaceutical products, characterized in that it comprises, in combination,

a plug with resilient means for adjustment to the inner face of the neck of a flask and an upper wall for stoppering the aperture or mouth of the bottle;

means for defining a position for pre-stoppering of the flask, before the stoppered position, in which the resilient adjustment means are not fully inside the neck of the bottle;

a protector joined to the plug which comprises a lateral wall, preferably of synthetic material, said lateral wall having on its inner face a projection for engagement with an annular protuberance located on the outer surface of the mouth of the bottle in the aforesaid stoppered position;

the aforesaid protector having an extension starting from the aforesaid projection in order to form, together with the transition zone between the neck and the main body of the flask, in the pre-stoppered position, a labyrinth closure with respect to the particles in suspension in the air surrounding the flask, the plug, in the pre-stoppered position, also being able to form another labyrinth closure together with the inner face of the annular rim of the mouth of the flask.

Preferably, in order to perform the required functions, the length of the extension of the lateral wall of the protector, from the upper point of the projection to its free end, will be equal to or greater than the travel of the stopper between the stoppered position and the pre-stoppered position.

The stopper of the present invention has the advantage of providing a labyrinth type closure against the particles in suspension in the air during the aforesaid processes of sterile measured filling, both in the stoppered position and in the pre-stoppered position. However, given their different physical states, the labyrinth closure does not prevent the sterilising steam in the autoclave from penetrating, thereby making it possible to sterilise the flask in the pre-stoppered position, minimising the time for which the inside of the flask is exposed to the ambient air during the measured filling process. On the other hand, the stopper of the present invention, by having the plug and the protector already joined to each other, provides the system with the following savings:

of logistical type: it is only necessary to provide and handle one part where two were handled before.

of economic type, in addition, since owing to the protuberance of the inside of the lateral wall of the protector it is not necessary to carry out the operation of closure by deformation of the metal capsule (beading).

This brings with it a significant saving of time, simplification of the equipment, and the reduction of overall costs.

Another additional advantage of the present invention lies in the fact that the conventional beading of the metal capsules generates particles which require the separation of the processes of measured filling and beading, an action which is unnecessary with the present invention.

The extension of the lateral wall may be defined as an extension of the projection of the lateral wall, as a straight extension of the lateral wall, as a conical extension below the projection of the lateral wall, or as a combination of a conical extension with a cylindrical end.

The stopper of the present invention in addition makes it possible to arrange an upper pull-off lid on the stopper. In this case, the protector may have an upper lid which gives access, via a window, to the plug made of rubbery material (rubber). This preferred embodiment will be directed especially to vials for sterile pharmaceutical products for intravenous administration.

As a consequence of all the above, the present invention also comprises the use of the stopper of the present invention in a process of sterile measured filling of flasks for pharmaceutical products which comprises, at least, the following steps:
placing the stopper on the flask, in a pre-stoppered position,
sterilisation in an autoclave, preferably with vacuum cycles,
preferably, a cooling step,
extraction of the stopper,
measured filling of the contents of the flask,
closure of the flask, placing the stopper in the stoppered position.

This method will be especially useful in the case where the flask is a vial for pharmaceutical products for intravenous administration.

For greater understanding of the invention, drawings of some embodiments of the present invention are appended by way of non-limiting example.

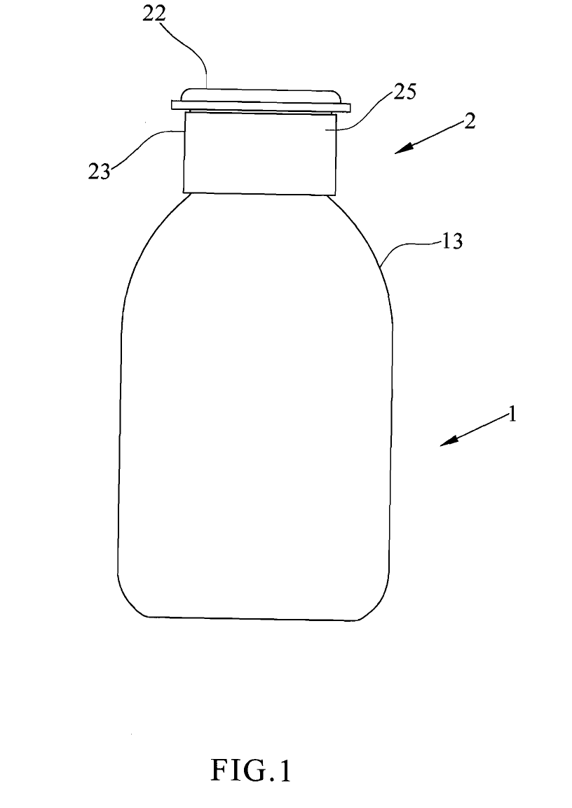

FIG. 1 shows a front view of a flask assembly for a pharmaceutical product, stoppered by means of a stopper according to the present invention.

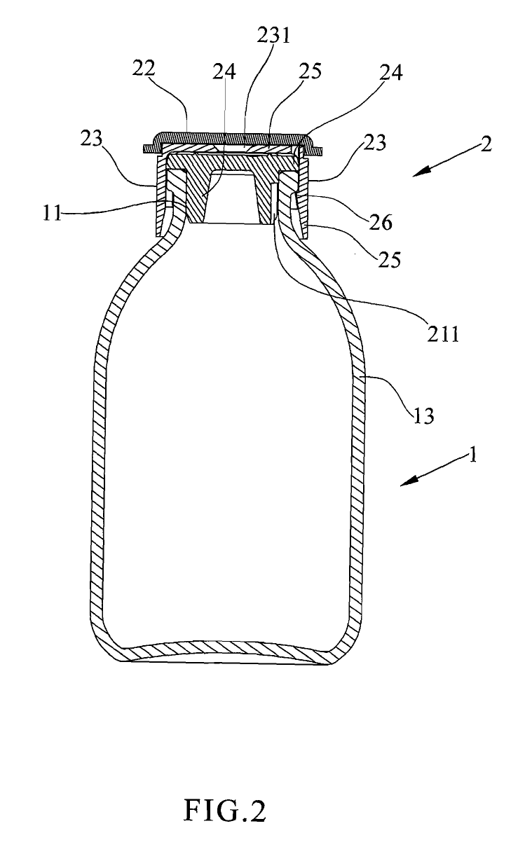

FIG. 2 shows a cross-section through the assembly of FIG. 1.

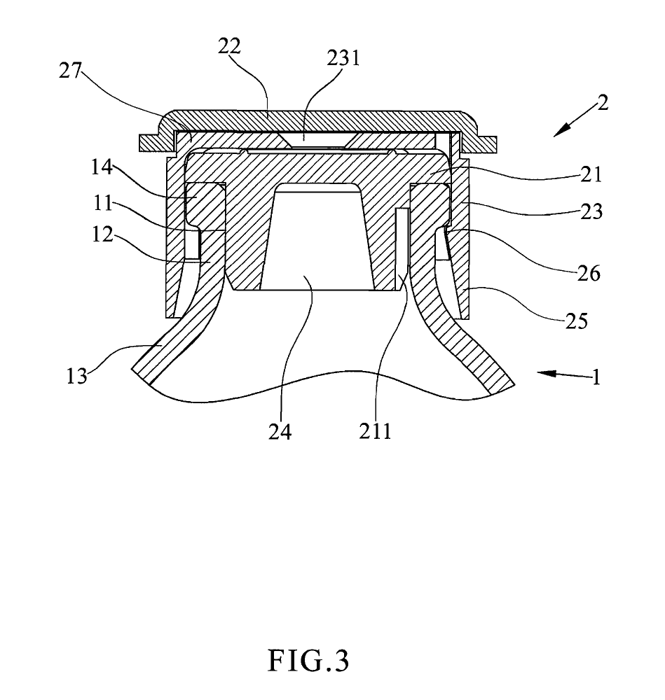

FIG. 3 shows a detail of the stopper of FIG. 2.

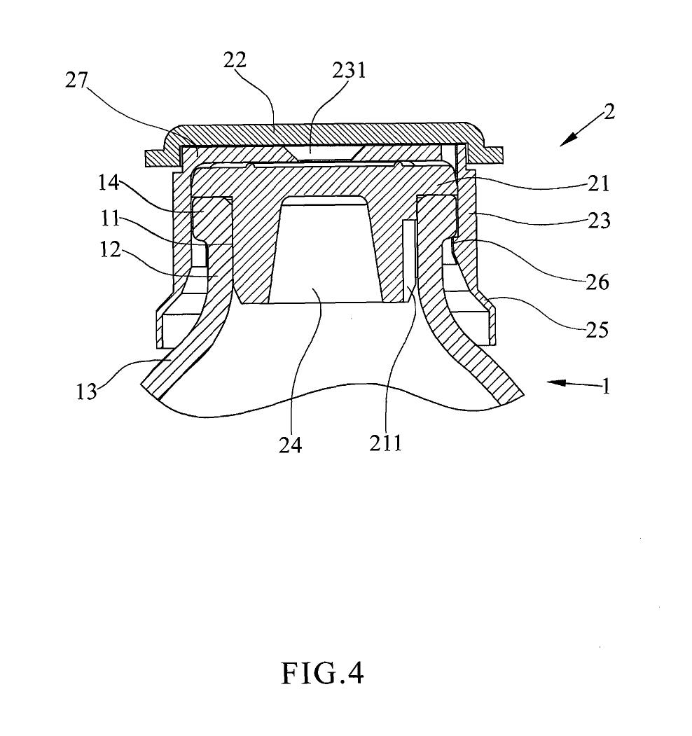

FIGS. 4, 5 and 6 show three alternative embodiments of the stopper according to the present invention.

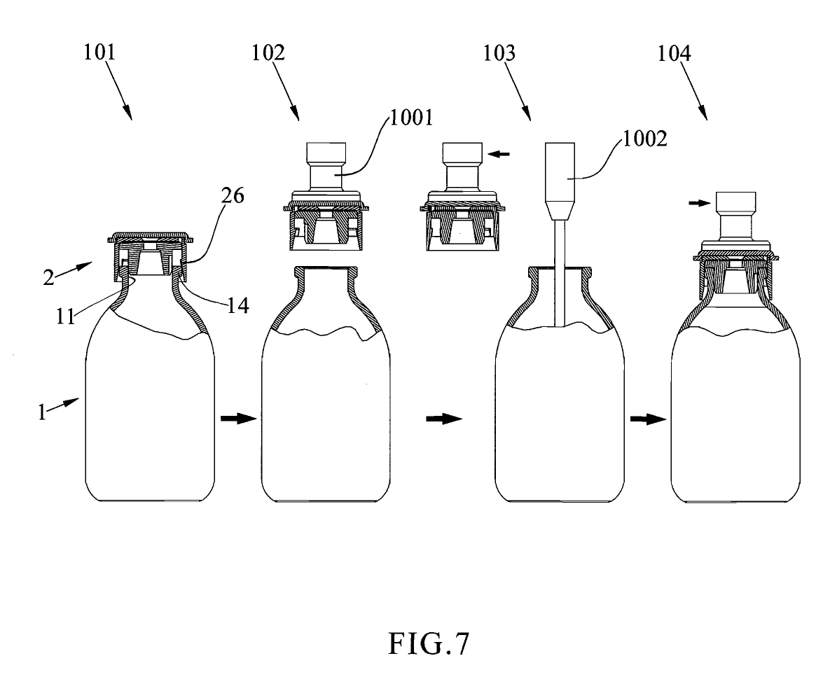

FIG. 7 shows diagrammatically a process of sterilisation and measured filling in which a stopper according to the present invention is used.

In FIGS. 1 to 3 an exemplary embodiment of the stopper 2 of the present invention can be seen in the stoppered position, closing a vial 1 for medicinal products.

As can be seen from the outside, the stopper 2 has an upper lid 22 which can be removed by being pulled off, and a lateral wall 23 which has an extension 25, which in the examples shown extends almost to touch the shoulder 13 (transition zone between the neck 12 and the main body of the vial 1), in such a way that the small gap between the shoulder 13 and the extension 25 forms a labyrinth closure for the ambient air, since it is complicated for any possible particles present in the ambient air to pass through the existing gap in a direction opposed to gravity.

Internally, the stopper 2 shown in the examples is composed of a plug 24, made of an elastomer, such as rubber. The plug 24 may have a special geometry which allows the stopper to rest in the mouth of the container (pre-stoppering) owing to a stepped contour (not shown). In addition, the plug 24 may have grooves 211 to permit, in the pre-stoppered position, the passage and emergence of the steam which will sterilise the container in the autoclave. The plug 24 also comprises an upper wall 21 for stoppering the bottle. This wall 21, made of rubbery material, makes it possible to be traversed by the needle of a syringe for the extraction of the pharmaceutical product from the interior without the need to open the stopper, which would expose the contents to the atmosphere.

The stopper shown also has a protector joined to the plug by insertion or assembly, or by any other means, of the plug inside the protector in a previous process not described herein. The protector comprises the aforesaid lateral wall 23, which will preferably be made of synthetic material, such as a plastics material, and the removable lid 22. Said lateral wall has on its inner wall a projection 26 for engagement with the annular protuberance 14 of the neck of the vial 1 in the mouth region. Said projection may be arranged along the entire perimeter of the inner face of the lateral wall 23 or at a specific number of points. Starting from the aforesaid projection 26, the lateral wall 23 has an extension 25 which, in the example of FIGS. 1 to 3, is in the shape of an extension of the projection and the free end of which extends so that it almost touches the shoulder 13 of the vial 1. In this way, a labyrinth closure is established against the contaminating particles which may be suspended in the ambient air.

In use, with the stopper in the closed position shown in FIGS. 1 to 6, it is possible to pull off the upper lid 22, which in this way acts as an upper pull-off enclosure. Beneath said enclosure 22 there is another upper lid 27 which has a window 231 which gives access to the upper wall 21 of the rubber plug 24, allowing it to be punctured by the needle of a syringe.

Similarly, in the example shown, the lower end of the plug 24 has an inclined/stepped wall which makes it possible to define a pre-stoppered position of the flask, in which the stopper rests, for example, on the inner edge of the mouth of the container 14 and the inner surface of the neck 11 of the container. In said position, it is preferable for the extension of the lateral wall 25 to have a length, from the upper point of the projection 26 to its free end, equal to or greater than the travel of the stopper 2 between the pre-stoppered position and the stoppered position. In this way, in the pre-stoppered position, the extension 25 and the annular protuberance of the mouth 14 of the container are close to each other, almost touching each other, so that in the pre-stoppered position the extension 25 of the lateral wall 23 also forms with the flask 1 a labyrinth closure with respect to the particles in suspension in the air.

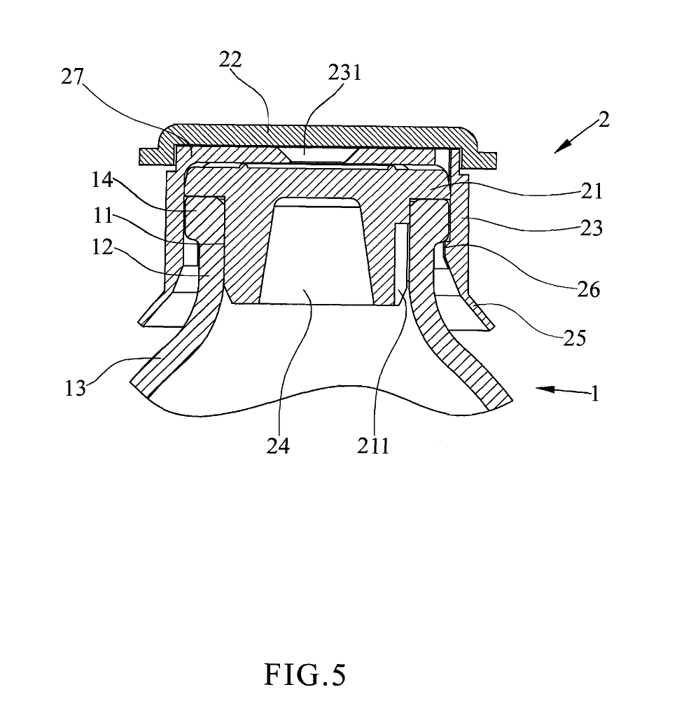

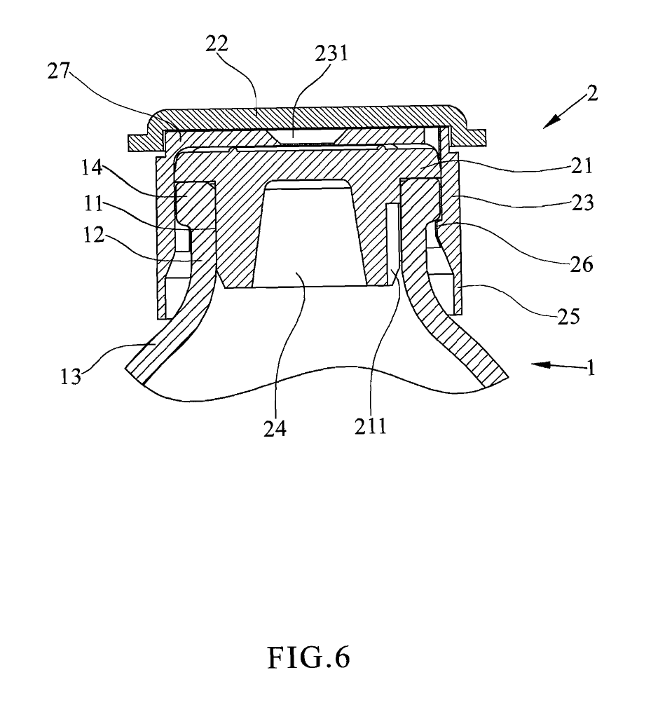

Naturally, the extension 25 of the lateral wall of the stopper 2 may be of various shapes. In FIG. 4, the extension 25 has a conical section with a cylindrical free end zone. In FIG. 5, the extension 25 is conical in shape on the outside. In FIG. 6, the extension 25 is cylindrical in shape on the outside and its free end is likewise cylindrical.

FIG. 7 shows an example of the use of a stopper 2 according to the present invention in a flask for pharmaceutical products, more specifically a vial 1.

The method shown includes a first step of placing the stopper 2 on the flask 1 in a pre-stoppered position; in this position a step 101 of sterilisation in an autoclave is then carried out. After the process of sterilisation in an autoclave and, optionally, cooling, the measured filling of the pharmaceutical product into the flask takes place, for which a first sub-step 102 of removal of the stopper (plug and protector) is carried out with a single implement 1001, followed by a second sub-step of filling 103 with a specific implement 1002 and then closure 104 of the stopper 2 on the flask 1 as far as the closed position, which can be performed, if desired, by means of the same implement 1001 which was used to separate the stopper from the flask. Once the container is closed, no beading operation is required, since the sealing thereof is obtained by clipping the protector onto the container.

It is therefore clear that the significant economy which is obtained in this method by using the stopper of the present invention, in comparison with methods of known type, in which use was made of a stopper (which included a metal capsule) and a protector as two separate parts, without any join.

Although a description has been given of some examples of preferred use of the stopper described, the invention is not necessarily limited to same. In particular, although applications have been shown in which the flask has a narrowed neck, said stopper may also be applied to flasks in which the neck has the same dimensions as the main body of the flask.

Although the invention has been described with respect to preferred exemplary embodiments, these should not be regarded as limiting the invention, which will be defined by the widest interpretation of the following claims.

1. A flask stopper, comprising, in combination:
a plug with resilient means for adjustment to the inner face of the neck of a flask and an upper surface for stoppering the opening or mouth of the flask;
means for defining a position for pre-stoppering of the flask, before the stoppered position, in which the resilient adjustment means are not fully inside the neck of the flask;
a protector joined to the plug which comprises a lateral wall, said lateral wall having on its inner face a projection for engagement with an annular protuberance located on the outer surface of the mouth of the container in the aforesaid stoppered position;
the aforesaid protector having an extension starting from the aforesaid projection to form, together with a transition zone between the neck and the main body of the flask, in the pre-stoppered position, a labyrinth closure with respect to the particles in suspension in the air surrounding the flask.

2. A stopper according to claim 1, wherein the length of the extension of the lateral wall of the protector, from the upper point of the projection to its free end, is equal to or greater than the travel of the stopper between the stoppered position and the pre-stoppered position.

3. A stopper according to claim 1, wherein the aforesaid extension is cylindrical in shape on the outside.

4. A stopper according to claim 1, wherein the aforesaid extension is conical in shape on the outside.

5. A stopper according to claim 1 wherein the aforesaid extension has a conical section with a cylindrical free end zone.

6. A stopper according to claim 1, further comprising a pull-off upper enclosure.

7. A stopper according to claim 1, wherein the protector has an upper lid with a window which gives access to the plug.

8. A stopper according to claim 1, wherein the plug is made of rubber.

9. A stopper according to claim 1, wherein the protector is made of synthetic material.

10. A stopper according to claim 1, wherein the means for defining a pre-stoppering position comprises a stepped contour on the plug.

11. A stopper according to claim 1, wherein the plug, in the pre-stoppered position, forms a labyrinth closure together with the inner face of the annular rim of the mouth of the flask.

12. A stopper according to claim 1, wherein the plug has a groove which, in the pre-stoppered position, permits the passage and emergence of vapor from inside the flask.

13. The use of a stopper according to claim 1 in a method of sterile measured filling of flasks of pharmaceutical products.

14. The use according to claim 13, wherein the method of sterile measured filling comprises the following steps:
placing the stopper on the flask, in the pre-stoppered position;
sterilisation in an autoclave;
measured filling of the pharmaceutical product into the flask;
closing the flask with the stopper, as far as its stoppered position.

15. The use according to claim 14, in which sterilisation takes place with the stopper on the flask, in its pre-stoppered position.

16. The use according to claim 13, wherein the flask is a vial for pharmaceutical products for intravenous administration.

17. The use according to claim 14, further comprising a cooling step between the sterilisation in an autoclave and the measured filling of the product.
